(19) United States
(12) Patent Application Publication
(10) Pub. No.: US 2025/0249311 A1
(43) Pub. Date: Aug. 7, 2025

(54) TRAINING DEVICE

(71) Applicant: Medica Medizintechnik GmbH, Hochdorf (DE)

(72) Inventor: Otto HOEBEL, Hochdorf (DE)

(73) Assignee: Medica Medizintechnik GmbH, Hochdorf (DE)

(21) Appl. No.: 19/184,644

(22) Filed: Apr. 21, 2025

Related U.S. Application Data
(63) Continuation of application No. PCT/EP2023/068220, filed on Jul. 3, 2023.

(30) Foreign Application Priority Data
Oct. 19, 2022 (DE) 10 2022 127 518.3

(51) Int. Cl. A63B 24/00; A63B 22/06; A63B 71/06; G16H 20/30; G16H 40/63

(52) U.S. Cl. CPC A63B 24/0062; A63B 22/0605; G16H 20/30; G16H 40/63; A63B 2024/0068; A63B 2024/0093; A63B 2071/0625; A63B 2230/062; A63B 2230/208

(57) ABSTRACT

A training device having a measuring system for detecting the performance achieved by a person undergoing training, and having a control system with an input channel for detecting a stress value and with an output channel for specifying the performance to be achieved. The control system is configured to carry out a method comprising the steps: specifying an initial stress value, detecting the value of the achieved performance when the initial stress value is reached, transferring the value of the achieved performance via the input channel, calculating the performance to be achieved for a training stress value as a function of a scale for perceived physical exertion, and outputting same via the output channel.

This nonprovisional application is a continuation of International Application No. PCT/EP2023/068220, which was filed on Jul. 3, 2023, and which claims priority to German Patent Application No. 10 2022 127 518.3, which was filed in Germany on Oct. 19, 2022, and which are both herein incorporated by reference.

### BACKGROUND OF THE INVENTION

### Field of the Invention

The invention relates to a training device having a measuring system for detecting a performance achieved by a person undergoing training, and having a control system with an input channel for detecting a stress value and with an output channel for specifying the performance to be achieved.

### Description of the Background Art

For training units on training devices that are used for rehabilitation purposes, in order to prevent injuries while still achieving an appropriate training effect it is important to ensure the correct training intensity for the person undergoing training. In the medical field, the correct training intensity may be determined using a scale for perceived physical exertion, in particular a linearized scale for perceived physical exertion, known as a Borg scale, for example. The Borg scale is used to determine the stress on a human based on his/her subjective perceived exertion. The stress levels of the Borg scale are greatly dependent on the human heart rate. To measure the heart rate, external sensors such as a pulse monitor have been used in the past, which over the entire training record the heart rate of the person undergoing training and in most cases visually output same, which makes the overall configuration more complex. It is also problematic that inexpensive, commercially available pulse monitors are not approved as a medical instrument due to the complexity, and use for medical purposes therefore poses problems and entails liability risks.

### SUMMARY OF THE INVENTION

It is therefore an object of the present invention to provide an option for monitoring a stress on the person undergoing training, without using external sensors.

In an example, the training device according to the invention is characterized in particular in that it has a measuring system for detecting the performance achieved by a person undergoing training, and a control system with an input channel for detecting a stress value and with an output channel for specifying the performance to be achieved. Furthermore, the control system is configured to carry out a method comprising the following steps: specifying an initial stress value, detecting the value of the achieved performance when the initial stress value is reached, transferring the value of the achieved performance via the input channel, calculating the performance to be achieved for a training stress value as a function of a scale for perceived physical exertion, and outputting the same via the output channel.

This results in the advantage of quicker and easier detection of a training stress value compared to detection using external sensors, such as a pulse monitor, by making use of the knowledge that is provided by the scale, which may be provided in particular by a Borg scale, namely, the Borg CR10 scale and/or the Borg CR20 scale.

It is thus necessary to calibrate the control system only once at the beginning, by associating the detected performance with the initial stress value to allow the control system to be subsequently used in a targeted manner for requesting the desired performance at the associated heart rate.

The training device is also able to specify, by means of the control system in a step e), a time-dependent performance profile for the training stress value. In addition, an output unit for signaling a deviation of the actual value from the target value of the training stress value, based on the performance profile, is present. It is thus possible to perform operations over an entire training unit without external sensors, and the person being trained receives via the output unit an indication that the training must be adapted in order to reach the target value. In an example, the output unit is designed as a screen, i.e. display screen, which makes it easier for the person undergoing training to understand and accept instructions given by the training device. In a further example, the output unit may also emit acoustic signals. The two above-mentioned examples are combinable.

To achieve the desired training effect, the control system is designed to change the rotational speed and/or the resistance for adapting the performance based on the performance profile. In an example, a measuring system that is provided for monitoring the physical stress on the person undergoing training when the initial stress value is reached may be associated with the training device. The stated measuring system may be designed to detect the heartbeat and/or oxygen consumption, and is used to ensure the use of the Borg scale as a redundant control instrument that does not have to monitor the entire training, but instead only warns before limit values are reached.

The features and feature combinations mentioned above in the description as well as the features and feature combinations mentioned below in the description of the figures and/or only shown in the figures may be used not only in the particular stated combination, but also in other combinations or alone without departing from the scope of the invention. Examples not explicitly shown or explained in the figures, but which follow and are producible from the described examples via separate feature combinations, are thus also regarded as encompassed and disclosed by the invention.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a perspective illustration of a training device,

FIG. 2 shows the individual perception of exertion according to the Borg CR10 scale for individuals with different performance capabilities; and

FIG. 3 shows the illustration of a time-dependent performance profile.

### DETAILED DESCRIPTION

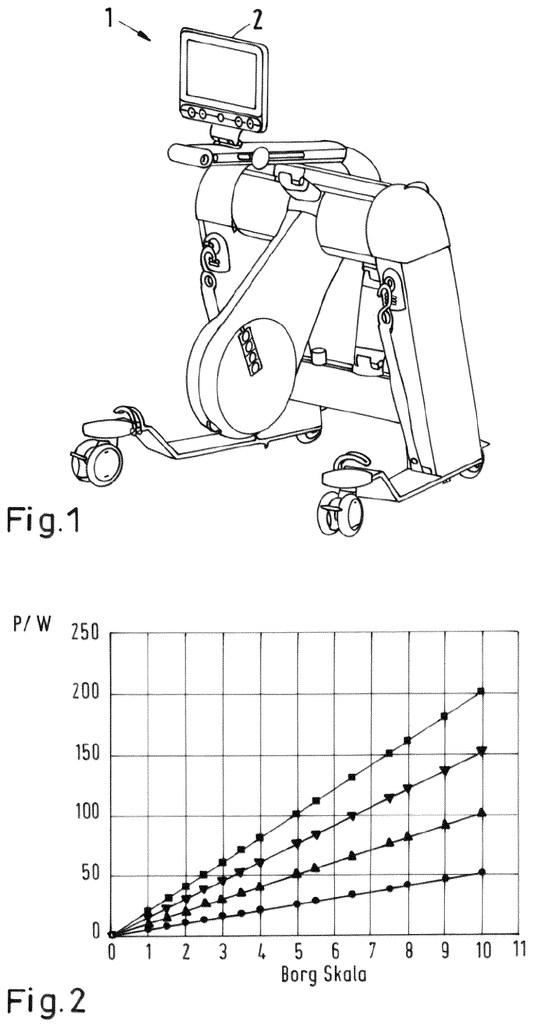

FIG. 1 shows a training device 1 in the example of an ergometer that is suitable for training of the arms and also the legs, and having a swivel unit 3 with a forced coupling for switching the extremities to be trained; however, the training device may also be designed as, for example, a treadmill, a stepper, a rowing machine, etc.

A measuring system for detecting the performance achieved by a person undergoing training, and a control system with an input channel for detecting a stress value and with an output channel for specifying the performance to be achieved are associated with the training device 1, the control system being configured to carry out a method comprising the steps: specifying an initial stress value, detecting the value of the achieved performance when the initial stress value is reached, transferring the value of the achieved performance via the input channel, calculating the performance to be achieved for a training stress value as a function of a scale for perceived physical exertion, and outputting the same via the output channel.

The scale for perceived physical exertion may be provided by a Borg scale, in particular the Borg CR10 scale and/or the Borg CR20 scale.

The Borg scale is a scale that has undergone rigorous scientific testing in order to determine the stress on a human based on the subjective perceived exertion (FIG. 2); the current linearized Borg CR10 scale reads as given below:
0=practically no exertion
0.5=extremely light (barely perceivable)
1=very light
2=light
3=moderate
4
5=heavy
6
7=very heavy
8
9,
10=extremely heavy (practically at maximum)

The Borg scale forms a linear relationship between the perceived exertion, the heart rate, and the individual stress level. FIG. 2 shows the individual perception of exertion for individuals with different performance capabilities. The values 0 through 2 of the Borg scale correspond to relaxation, the interval 2 through 3 corresponds to light stress, the interval 3 through 5 corresponds to moderate stress, the interval 5 through 7 corresponds to heavy stress, and the interval 7 through 10 corresponds to maximum stress. The following table provides a further scaling of the values according to the historically oldest scale, namely, the Borg CR20 scale, which, the same as for other exertion scales, is convertible into the current Borg scale:

| Borg scale | | Perceived |
|---|---|---|
| Value | Pulse | exertion |
| 6 | 60 | No exertion |
| 7 | 70 | Extremely light |
| 8 | 80 | |
| 9 | 90 | Very light |
| 10 | 100 | |
| 11 | 110 | Light |
| 12 | 120 | |
| 13 | 130 | Somewhat heavy |
| 14 | 140 | |
| 15 | 150 | Heavy |
| 16 | 160 | |
| 17 | 170 | Very heavy |
| 18 | 180 | |
| 19 | 190 | Extremely heavy |
| 20 | 200 | Maximum exertion |

The table also shows a linear relationship between the values of the Borg scale and the heart rate. The perceived exertion is categorized in the right column, so that here as well it is possible, based on the subjective perceived exertion, to draw conclusions concerning a corresponding value in the Borg scale.

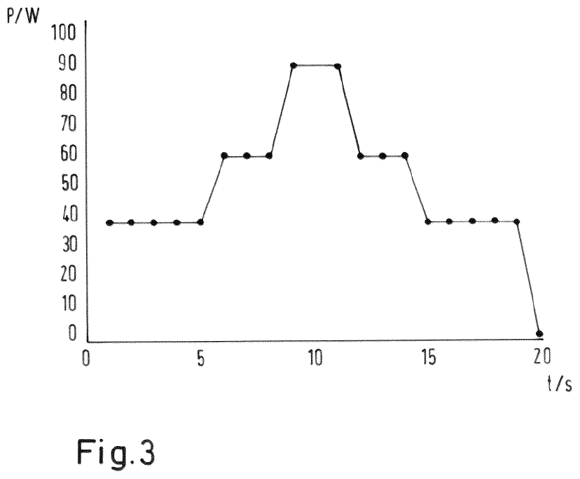

FIG. 3 shows an example of a time-dependent performance profile which is specified by the control system. The graphic illustrated in FIG. 3 represents by way of example the target values of the training stress value at any point in time on the training unit. An output unit for signaling a deviation of the actual value from the target value of the training stress value is provided. This output unit is designed as a screen 2 in the present invention. An acoustic signal and/or a vibration signal may also be provided, possibly as a supplement.

The control system of the training device 1 is designed to change the rotational speed and/or the resistance for adapting the performance, based on the performance profile. The training device 1 also has a measuring system for monitoring the physical stress on the person undergoing training when the initial stress value is reached. The measuring system is designed to detect the heartbeat and/or oxygen consumption.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A training device comprising:
a measuring system to detect a performance achieved by a person undergoing training; and
a control system with an input channel for detecting a stress value and with an output channel to specify the performance to be achieved, the control system being configured to carry out a method comprising:
specifying an initial stress value;
detecting the value of the achieved performance when the initial stress value is reached;
transferring the value of the achieved performance via the input channel;
calculating the performance to be achieved for a training stress value as a function of a scale for perceived physical exertion; and
outputting the calculated performance to be achieved via the output channel.

2. The training device according to claim 1, wherein the scale for perceived physical exertion is provided by a Borg scale.

3. The training device according to claim 2, wherein the Borg scale is provided by the Borg CR10 scale and/or the Borg CR20 scale.

4. The training device according to claim 1, wherein the control system specifies a time-dependent performance profile for the training stress value.

5. The training device according to claim 4, wherein an output unit is provided for signaling a deviation of the actual value from the target value of the training stress value, based on the performance profile.

6. The training device according to claim 5, wherein the output unit is a display screen.

7. The training device according to claim 5, wherein the output unit emits acoustic signals.

8. The training device according to claim 1, wherein the control system is designed to change a rotational speed and/or a resistance for adapting the performance based on the performance profile.

9. The training device according to claim 1, wherein a measuring system is provided for monitoring the physical stress on the person undergoing training when the initial stress value is reached.

10. The training device according to claim 9, wherein the measuring system detects a heartbeat and/or oxygen consumption.
